(19) United States
(12) Patent Application Publication
(10) Pub. No.: US 2024/0263163 A1
(43) Pub. Date: Aug. 8, 2024

(54) CELL-FREE REGIONS BY LABEL-FREE MAGNETIC EXCLUSION

(71) Applicant: McMaster University, Hamilton (CA)

(72) Inventors: Tamaghna Gupta, Hamilton (CA); Rakesh P. Sahu, Hamilton (CA); Ishwar K. Puri, South Pasadena, CA (US)

(21) Appl. No.: 18/565,957

(22) PCT Filed: Jun. 1, 2022
(86) PCT No.: PCT/CA2022/050875

§ 371 Date: Nov. 30, 2023

Related U.S. Application Data
(60) Provisional application No. 63/202,208, filed on Jun. 1, 2021.

(51) Int. Cl. C12N 13/00; C12M 1/32; C12M 1/42; C12N 5/071

(52) U.S. Cl. CPC C12N 13/00; C12M 23/12; C12M 35/06; C12N 5/0688; C12N 2529/00; C12N 2533/90

(57) ABSTRACT

This application relates to methods of generating cell/particle-free regions in a cellular/particle aggregate using label-free magnetic manipulation. For example, the method may include suspending cells/particles in a paramagnetic culture medium, the cells/particles being diamagnetic; seeding the cells/particles and the paramagnetic culture medium into microwells; and placing the microwells over an array of magnet pairs where each magnet pair creates a magnetic field gradient. The magnetic susceptibility difference and the magnetic field gradient drive the cells/particles towards a spaced region between magnets in the magnet pair having a lowest magnetic field strength to create a cell/particle aggregate with a cell-free/particle-free area where the cell/particle aggregate surrounds the cell-free/particle-free area.

### FIELD

This application claims the benefit of U.S. Provisional Patent Application No. 63/202,208 filed Jun. 1, 2021; the entire contents of Patent Application No. 63/202,208 is hereby incorporated by reference.

### FIELD

The present application relates to the field of particle assembly, bioprinting, and in particular, to label-free magnetic bioprinting.

### BACKGROUND

The development and maintenance of multicellular organisms require cell migration. Biological processes, such as embryonic morphogenesis, development of the nervous system, tissue homeostasis, and immune surveillance involve orchestrated cell movement in response to various biochemical and biophysical cues^{1,2}. Signaling molecules regulate cell migration under normal physiological conditions by controlling cell adhesion and cytoskeleton organization^{1,3,4}. Conversely, pathological conditions such as metastatic cancer, vascular disease, and chronic inflammation are associated with unregulated cell migration, contributing to disease progression^{4,5}.

The wound healing assay is a commonly used in vitro method to study cell migration, cell interactions, and the underlying mechanisms⁶⁻¹⁰. The conventional “scratch” assay creates a cell-free area or scratch in a cell monolayer using a pipette tip or a pin tool^{10,11}. Cells from intact surrounding regions migrate and repopulate the cell-free region over time. The migration rate is measured by observing the decrease in the cell-free area at regular intervals. While the scratch assay is simple and inexpensive, it requires 24-48 hours to form a cell monolayer¹¹. Moreover, by physically scratching the cell layer with a pipette tip or microneedle, cell-free regions of inconsistent shapes and sizes are created^{10,12,13}. This process also damages the underlying plastic surface or extracellular matrix (ECM) substrate, which can significantly affect cell migration^{10,12,13}.

The electric cell-substrate impedance sensing (ECIS) technique, on the other hand, produces consistent cell-free regions without mechanically disrupting the cell layer^{10,12}. In ECIS, a confluent cell layer is formed over a small gold film electrode placed at the bottom of a tissue culture well. Electric pulses are applied to induce cell death at the electrode surface, which creates a well-defined region devoid of cells¹⁴. However, dead cell fragments can remain on top of the electrode and interfere with cell migration^{12,15} Furthermore, the electric pulses can potentially injure the cells surrounding the electrode, thereby affecting their migration¹⁰.

In contrast to the scratch assay and ECIS, the physical exclusion technique creates cell-free areas of reproducible sizes and shapes with minimal cellular damage^{12,16}. This method employs physical barriers, such as plastic inserts or silicone stoppers to prevent cells from settling in a pre-defined area. However, it requires 12-20 hours to form confluent cellular regions around the barrier^{16,17}. Commercial inserts are also expensive and unsuitable for multiple uses¹⁶.

Magnetic levitation is another in vitro technique used to form cell clusters in a migration assay¹⁸. This method is based on the cellular uptake of biopolymer-encapsulated magnetic iron oxide nanoparticles^{18,19}. Once magnetized with nanoparticles, a magnetic field is used to form aggregates. Similar to conventional techniques, magnetic levitation is time-consuming as it requires overnight incubation of cells with the magnetic nanoparticles. Further, their interactions with nanoparticles can be detrimental to cell functionality²⁰. There is currently a significant need for improved cell migration assays that are reproducible, rapid, inexpensive and easy to use.

### SUMMARY

In one aspect, at least one embodiment of the present application discloses a label-free “magnetic exclusion” technique by which magnetic fields, instead of physical barriers, are employed to create cell-free regions. Paramagnetic suspensions of human bronchial epithelial (HBEC3 KT) cells are added to tissue culture wells placed on a surface, such as a coaxially arranged magnets, such as cylinder magnets that are located within ring magnets, for example. Driven by the susceptibility difference and magnetic field gradient, the cells assemble into annular aggregates within a few hours. The formation of the annular aggregates on tissue culture-treated (TCT) and ECM-coated well surfaces is demonstrated and the cell-free area closure under different epidermal growth factor (EGF) concentrations is described. A reaction-diffusion equation-based mathematical model is employed to analyze the dynamics of the cell-free area closure^{27,28}. Transcriptome analysis, live/dead, and metabolism assays are performed to investigate the effects of the paramagnetic solution on cell viability and functionality. No significant changes in cell viability, metabolism, and transcriptional profiles are observed due to the paramagnetic agent exposure.

It has been found that the magnetic exclusion technique described in accordance with the teachings herein is generally rapid, inexpensive, and/or easy to use. In at least one embodiment, a cell or particle aggregation process described herein may be performed with or without using any physical inserts and can hence be easily automated. The device fabrication is generally straightforward, and in at least one embodiment ring-cylinder magnet pairs may be used which may be tailored to standard high-density microwell plates. The label-free method doesn't require any scratching tips. The size and shape of the cell-free regions may be defined based on the shapes of the magnets used in the array of magnet pairs and on the magnet dimensions as well as the manufacturing tolerances of the magnets which may be on the order of about ±0.1 mm. Thus, the methods described herein may produce one or more cellular or particle aggregates that have high reproducibility, making the method amenable for various purposes such as, but not limited to, high throughput drug screening in preclinical research, for example.

In one aspect, in accordance with the teachings herein there is provided at least one embodiment of a method for creating cell-free regions by magnetic exclusion, wherein the method comprises:
a) suspending cells in a paramagnetic culture medium, the cells being diamagnetic, and
b) seeding the cells and the paramagnetic culture medium into microwells to create a magnetic susceptibility difference and placing the microwells on an array of magnets that create magnetic field gradients,
   wherein the magnetic susceptibility difference and the magnetic field gradient drive the cells towards an annular region of the lowest field strength, and thus creating the cell-free area.

In at least one embodiment, the method comprises producing the annular cell aggregate with or without a spacer.

In another aspect, in accordance with the teachings herein, there is provided a method of generating cell-free regions in a cellular aggregate using label-free magnetic manipulation, wherein the method comprises: suspending cells in a paramagnetic culture medium, the cells being diamagnetic create a magnetic susceptibility difference; seeding the cells and the paramagnetic culture medium into microwells; and placing the microwells over an array of magnet pairs where each magnet pair creates a magnetic field gradient, wherein the magnetic susceptibility difference and the magnetic field gradient drive the cells towards a spaced region between magnets in the magnet pair having a lowest magnetic field strength to create the cellular aggregate with a cell-free area where the cellular aggregate surrounds the cell free-area.

In at least one embodiment, each magnet pair includes an outer magnet and an inner magnet, and the magnetic field gradient is from both each magnet to the spaded region.

In at least one embodiment, the inner magnet is approximately coaxially aligned with the outer magnet.

In at least one embodiment, the method comprises using spacers in the magnet pairs to hold inner magnets in place with respect to outers magnets.

In at least one embodiment, the outer magnet comprises a ring magnet and the inner magnet comprises a cylinder magnet.

In at least one embodiment, an internal wall of the outer magnet is selected to have a desired shape to generate the cellular aggregate where the cell-free area has the desired shape.

In at least one embodiment, the desired shape includes a rectangular shape, a square shape, an elliptical shape or a circular shape.

In at least one embodiment, the array of magnet pairs is disposed on a support plate.

In at least one embodiment, the microwells are provided in a well plate, a cell culture plate, a micro titer plate or an individual cuvette.

In at least one embodiment, the method comprises incubating the culture medium for an incubation period after placing the microwells over the array of magnet pairs.

In at least one embodiment, the method may comprise performing at least one wash of the cellular aggregate after the incubation period.

In at least one embodiment, the paramagnetic culture medium is prepared using a paramagnetic salt solution.

In at least one embodiment, the microwells have synthetic and/or natural extracellular matrix (ECM) coated well surfaces upon which the annular cell aggregates can be produced.

In at least one embodiment, the microwells have tissue culture treated surfaces or collagen I-fibronectin coated surfaces.

In at least one embodiment, the cells are selected from at least one of adherent cell lines, non-adherent cell lines, primary cells, and stem cells.

In at least one embodiment, the cells comprise different cells lines and/or different cell types that may be co-cultured. For example, the different cell types may be epithelial cells, fibroblasts, muscle cells and other possible cell types.

In at least one embodiment, the method comprises forming the cellular aggregate with more than one layer of cells using similar or different cell types.

In at least one embodiment, the method comprises forming layers of the cellular aggregate on top of one another so that the layers are adjacent to one another or separated by a natural ECM or a synthetic ECM.

In at least one embodiment, the magnets comprise manufacturing tolerances of about ±0.1 mm for creating cellular aggregates with uniform cell-free areas having increased reproducibility to facilitate high throughput assays.

In at least one embodiment, the method comprises generating cellular aggregates for use in cell migration assays, 3D cell invasion assays, wound healing assays, study cell interactions, drug discovery, drug screening, and/or disease models.

In at least one embodiment, the method comprises using cellular aggregates for fabrication of smooth muscle rings for asthma models or Inflammatory Bowel Disease models.

In another aspect, in accordance with the teachings herein, there is provided at least one embodiment of a method of generating particle-free regions in a particle aggregate using label-free magnetic manipulation, wherein the method comprises: suspending particles in a paramagnetic culture medium to create a magnetic susceptibility difference, the particles being diamagnetic; seeding the particles and the paramagnetic culture medium into microwells; and placing the microwells over an array of magnet pairs where each magnet pair creates a magnetic field gradient, wherein the magnetic susceptibility difference and the magnetic field gradient drive the particles towards a spaced region between magnets of the magnet pair having a lowest magnetic field strength to create the particle aggregate with a particle-free area where the particle aggregate surrounds the particle free-area.

In at least one embodiment, the particle aggregates are formed to create electrodes.

In at least one embodiment, the particles comprise reduced graphene oxide and/or copper.

In at least one embodiment, the particles comprise diamagnetic nanoparticles.

In another aspect, in accordance with the teachings herein, there is provided at least one embodiment of a kit comprising a paramagnetic agent, an array of magnet pairs for applying an external magnetic field gradient to at least one receptacle.

In at least one embodiment, the magnet pairs comprise an outer magnet and an inner magnet that is disposed within the outer magnet with a spaced region therebetween.

In at least one embodiment, the kit further comprises spacers that are shaped for placement within the magnet pairs.

In at least one embodiment, the kit further comprises a cell culture plate, a cuvette or a micro titer plate that provides the at least one receptacle.

In another aspect, in accordance with the teachings herein, there is provided at least one embodiment of an apparatus for generating element-free regions in an element aggregate using label-free magnetic manipulation, wherein the apparatus comprises: a plurality of microwells that are seeded with elements in a paramagnetic culture medium to create a magnetic susceptibility difference, the elements being diamagnetic, and an array of magnet pairs where each magnet pair creates a magnetic field gradient, wherein the plurality of microwells with the elements and paramagnetic culture medium are disposed over the array of magnet pairs, and wherein the magnetic susceptibility difference and the magnetic field gradient drive the elements towards a spaced region between magnets in the magnet pair having a lowest magnetic field strength to create the element aggregate with an element-free area where the element aggregate surrounds the particle free-area.

In at least one embodiment, the elements comprise cells or particles.

It will be appreciated that the foregoing summary sets out representative aspects of embodiments to assist skilled readers in understanding the following detailed description. Other features and advantages of the present application will become apparent from the following detailed description taken together with the accompanying drawings. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIGS. 1A-1D show formations of annular HBEC3 KT monolayers in one example embodiment of the application in which FIG. 1A. shows a schematic of the coaxially arranged ring-cylinder magnet array with an N-N orientation and a 96 well plate is placed on the array, with the center of each ring-cylinder magnet arrangement aligned with a well; FIG. 1B shows a top view of a single ring-cylinder magnet arrangement with magnetic flux density distribution at the surface of the well bottom and a spacer is inserted between the ring and cylinder magnets to maintain a consistent gap; FIG. 1C shows an Illustration of the different steps to form an annular aggregate within 3 h where the washing step removes the loose cell aggregates, resulting in a monolayer; and FIG. 1D shows fluorescent stitched images of an annular monolayer assembled on (i) tissue culture-treated and (ii) collagen I and fibronectin-coated surfaces where the cells are stained with Calcein AM and the brightfield image shows the cell organization in an annular section.

FIG. 2 shows brightfield images (contrast-enhanced) of an annular monolayer section before and after the washing step where the washing step removes the loose cell aggregates, resulting in a monolayer and the scale bar is 250 μm.

FIGS. 3A-3B show the distribution of effective magnetophoretic force and simulated cell positions in another example embodiment of the application in which FIG. 3A (i) shows a cross-sectional view of the ring-cylinder magnet arrangement in the r-z plane with the computational domain for the particle tracing simulation is marked as abcd; FIG. 3A (ii) shows the effective magnetophoretic force (F_{m,eff}) distribution in the computational domain where the bold black line shows the levitation heights and the inset shows that (F_{m,eff}) is directed towards the magnetic field minima; and FIG. 3B shows simulated cell positions at different times where the cells are levitated in the regions where F_{m,z} the z component of the magnetophoretic force, is balanced by the net gravitational force.

FIG. 4 shows fluorescent image of an annular HBEC3 KT monolayer formed by the magnetic exclusion technique after 85 min incubation and the arrows show the isolated cell clusters.

FIGS. 5A-5E show the effect of residual Gd³⁺ on cell viability and functionality in another example embodiment of the application where FIG. 5A shows residual Gd³⁺ ions detected in the wells containing annular monolayers (N=3); FIG. 5B shows the effect of residual Gd³⁺ on cell viability, measured using a live/dead assay kit where fluorescent images show live (blue) and dead (green) cells at different times and cell monolayers formed by gravitational settling are used as controls (N=3, technical replicates=3) and the scale bar is 500 μm; FIG. 5C shows viable cell counts obtained from the live/dead fluorescent images using ImageJ; FIG. 5D shows the effect of residual Gd³⁺ on cell metabolism, measured using a resazurin-based assay (N=3, technical replicates=3); and FIG. 5E shows the effect of residual Gadolinium on the transcriptional profiles of HBEC3 KT cells (N=4) where each grey dot represents the expression level of a gene with levels in control samples (Y-axis) plotted against expression levels in Gadolinium exposed samples (X-axis) and the absence of statistically significant genes that deviate in expression levels between control and Gadolinium implies insignificant impacts on transcriptional profiles.

FIGS. 6A-6G show the effect of surface coating and EGF concentrations on cell migration in another example embodiment of the application in which FIGS. 6A-6B) show fluorescent images of the cell-free areas at different times for different EGF concentrations and surface conditions where the annular monolayers are formed by the magnetic exclusion technique and the white dotted line shows the boundary of a cell-free area at 0 h; FIG. 6C shows closure of the cell-free regions of annular monolayers formed on tissue culture-treated surfaces (N=3, technical replicates=3); FIG. 6D shows closure of the cell-free regions of annular monolayers formed on collagen I and fibronectin-coated surfaces (N=3, technical replicates=3); FIG. 6E shows a schematic of a 3D-printed annular insert used to form annular monolayers by gravitational settling; FIG. 6F shows fluorescent stitched images of annular monolayers formed by adding (i) ˜50,000 and (ii) ˜70,000 HBEC3 KT cells to the annular inserts where the inserts are removed after 9 h incubation; And FIG. 6G shows a comparison of cell-free area closure of annular monolayers assembled by the magnetic exclusion and gravitational settling techniques (N=1, technical replicates=5) and the scale bar is 1 mm.

FIG. 7 shows a fluorescent image of an annular HBEC3 KT monolayer formed by gravitational settling after 3 h incubation.

FIGS. 8A-8D show EGF dependent cell migration in another example embodiment of the application in which FIG. 8A shows circular tracks superimposed on a fluorescent image of an annular monolayer where the tracks are used to count cells within the domain 0≤r≤Rₘₐₓ at different times and here, r=0 is shown by the ‘+’ sign and the cell counts are used to generate cell density profiles for 0 ng mL⁻¹ shown in FIG. 8B, 0.16 ng mL⁻¹ shown in FIG. 8C, and 0.8 ng mL⁻¹ EGF shown in FIG. 8D (N=1, technical replicates=3).

FIGS. 9A-9B show validation of the numerical model in another example embodiment of the application where the predicted cell density profiles (dotted lines) in FIG. 9AA Replicate 1 and FIG. 9B Replicate 2 where for model cross-validation, the values of D₀, D₁ and m obtained from Replicate 1 are used to fit the experimental data of Replicate 2 and the cell density profile at 0 h provides the initial condition to the numerical model, and hence, numerical fits are not obtained for the 0 h experimental data.

### DETAILED DESCRIPTION OF THE EXAMPLE EMBODIMENTS

### Definitions

Unless otherwise indicated, the definitions and embodiments described in this and other sections are intended to be applicable to all embodiments and aspects of the present application herein described for which they are suitable as would be understood by a person skilled in the art.

Various embodiments in accordance with the teachings herein will be described below to provide examples of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements or steps. In addition, numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending on the context in which these terms are used. For example, the terms coupled or coupling can have a mechanical, electrical, magnetic or communicative connotation. For example, as used herein, the terms coupled or coupling can indicate that two elements or devices can be directly connected to one another or connected to one another through one or more intermediate elements or devices via an electrical element, an electrical signal, a magnetic field or a mechanical element depending on the particular context.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of the modified term, such as of at least ±1%, ±2%, ±5% or ±10% of the modified term if this deviation would not negate the meaning of the word it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as ±1%, ±2%, ±5%, or ±10%, for example.

Various terms used throughout the present description may be read and understood as follows, unless the context indicates otherwise: singular articles and pronouns as used throughout include their plural forms, and vice versa; similarly, gendered pronouns include their counterpart pronouns so that pronouns should not be understood as limiting anything described herein to use, implementation, performance, etc. by a single gender. Further definitions for terms may be set out herein; these may apply to prior and subsequent instances of those terms, as will be understood from a reading of the present description. For example, as used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination and therefore the wording “and/or” is intended to represent an inclusive-or. That is, “X and/or Y” is intended to mean X or Y or both X and Y, for example. As a further example, “X, Y, and/or Z” is intended to mean X or Y or Z or any combination thereof. In effect, this term means that “at least one of” or “one or more” of the listed items is used or present.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

### EXAMPLES

Label-free magnetic manipulation is a rapid and versatile cell assembly technique by which cells are suspended in a solution of higher magnetic susceptibility which provides a paramagnetic medium. When exposed to an inhomogeneous magnetic field, a resulting magnetophoretic force drives the paramagnetic medium toward regions of highest magnetic field strength, displacing the cells toward regions of lowest field strength, where they form clusters²¹. The result is that the paramagnetic medium is mixed into a solid-fluid suspension, leading to a difference between the magnetic susceptibilities of the solid and its surrounding medium, which induces differential magnetic forces on the suspension constituents. When an external magnetic field is applied on such a cell-medium system, a cell behaves as a diamagnetic material that migrates towards the region of lower magnetic field strength. This method has been used to levitate cells, and form spheroids²²⁻²⁶. However, the formation of cellular aggregates for cell migration assays have not yet been demonstrated with this method.

The following non-limiting examples are illustrative of the present application.

### Example 1

### Materials and Methods

In an experimental study, a cell culture was created by culturing immortalized human bronchial epithelial cells HBEC3 KT (ATCC) in Keratinocyte-SFM (serum-free medium) supplemented with 50 μg mL⁻¹ bovine pituitary extract (BPE), 0.8 ng mL⁻¹ epidermal growth factor (EGF), and 1% penicillin/streptomycin (ThermoFisher). The cells were maintained at 37° C. and 5% CO₂ in a humidified atmosphere.

The magnetic exclusion technique was applied to form annular HBEC3 KT monolayers in which HBEC3 KT cells were trypsinized at ˜80% confluency. A 96-well flat-bottom TCT plate (IBIDI) was placed on an array of coaxially arranged ring and cylinder magnets (N52 grade, Zigmyster Magnets). The stock gadolinium-based paramagnetic solution (1 M) was diluted to 25×10⁻³ M using the serum-free culture medium. A 250 μL suspension of ˜50,000 cells in 25×10⁻³ M paramagnetic solution was added to the wells. After a 3 h incubation period (37° C. and 5% CO₂ in a humidified atmosphere), the well plate was removed from the magnet array. The paramagnetic solution was removed, and the wells were washed twice with 200 μL culture medium. Next, the formation of annular HBEC3 KT monolayers on collagen I-fibronectin surfaces was demonstrated. Tissue culture wells were plasma treated to enhance the attachment of the ECM proteins to the well surfaces. A 300 μL solution of collagen I (500 μg mL⁻¹) and fibronectin (12 μg mL⁻¹) was added to the wells. The well plate was incubated at 4° C. overnight, and subsequently, the coated wells were rinsed with phosphate-buffered saline (PBS). Annular HBEC3 KT monolayers were assembled magnetically on the coated surfaces. A fluorescent dye solution of 2 μM Calcein AM and Hoechst 33342 (2 drops per mL) was used to stain the monolayers. Brightfield and fluorescent images were taken using a 10× objective of an EVOS M7000 inverted microscope (ThermoFisher). Excitation/emission wavelengths of 470/525 nm and 357/447 nm were used for Calcein AM and Hoechst 33342, respectively.

The experimental study assessed the impact of gadolinium ions on the process by detecting of residual gadolinium ions. HBEC3 KT annular aggregates were formed in a 96-well plate using the magnetic exclusion technique. The wells were washed twice with 200 μL PBS. The cells were trypsinized, and the suspensions were pooled together in a microcentrifuge tube. For control samples, ˜50,000 HBEC3 KT cells were seeded into wells containing only the culture medium and incubated for 3 h. The amount of Gd³⁺ in the samples was detected using inductively coupled plasma mass spectrometry (Agilent 7700 series).

The experimental study also involved performing live/dead assays. A 400 μL suspension of ˜5000 HBEC3 KT cells in 25×10⁻³ M paramagnetic solution was added to the wells of a 48-well TCT plate. After a 3 h incubation period, the paramagnetic solution was replaced with the culture medium. As controls, an equal number of cells were seeded into wells containing only the culture medium. The cell viability was assessed using the NucBlue Live and NucGreen Dead nuclear acid stains (ReadyProbes, ThermoFisher). Fluorescent images were taken at 24, 48, 96, and 144 h. Excitation/emission wavelengths of 357/447 nm and 470/525 nm were used for NucBlue and NucGreen, respectively. The quantification of live and dead cells was performed using ImageJ Fiji.

The experimental study also involved performing a metabolism assay. A 250 μL suspension of ˜4000 HBEC3 KT cells in 25×10⁻³ M paramagnetic solution was added to the wells of a 96-well plate. Following a 3 h incubation period, the paramagnetic solution was replaced with the culture medium. An equal number of cells suspended in the culture medium were added to the wells as controls. A resazurin-based assay (Presto Blue HS, ThermoFisher) was used to analyze cell metabolism at 24, 48, 96, and 144 h. The fluorescence intensity was measured at multiple spots within each well using excitation/emission wavelengths of 560/590 nm (SpectraMax i3, Molecular Devices). Blank wells containing only culture medium were used to account for background fluorescence. The average fluorescence intensity of the blanks was subtracted from the average fluorescence value obtained from each well of the Gadolinium and control groups.

The experimental study also involved performing transcriptome analysis. A 1.5 mL suspension of ˜500,000 HBEC3 KT cells in 25×10⁻³ M paramagnetic solution was added to the wells of a 12-well TCT plate. Following a 3 h incubation period, the paramagnetic solution was replaced with the culture medium. After 24 h, total RNA was isolated and purified using RNeasy Mini Kit (Qiagen) as per the manufacturer's protocol. The RNA concentration and purity were measured using a spectrophotometer (NanoDrop, ThermoFisher). Transcriptome analysis was performed using Clariom S microarray gene chips (ThermoFisher).

The experimental study also involved assessing annular HBEC3 KT monolayer formation by gravitational settling. Annular inserts were 3D printed with a Formlabs Form 2 printer using a clear resin (RS-F2-GPCL-04). The inserts were attached to the well surfaces of a 12-well plate using patterned double-sided silicone adhesive tapes. The well plates containing the inserts were sterilized with UV radiation. A 100 μL suspension of ˜50,000 HBEC3 KT cells in culture medium was added to the inserts. After a 3 h incubation period, the inserts were removed, and the wells were washed twice with 500 μL culture medium.

Cell counting and geometric measurements were automatically determined in the experimental study from fluorescent images with ImageJ Fiji. The watershed segmentation algorithm (Process—Binary—Watershed) was used to distinguish cells in high cell density regions. The brightfield image contrast was adjusted using the Enhance Local Contrast (CLAHE) ImageJ plugin to measure the cell-free area. The Sobel edge detection algorithm (Process—Find edges) was used to highlight the boundaries of the monolayers. Next, image smoothing was performed using the Gaussian Blur filter (Process—Filter—Gaussian Blur). The cell-free areas were measured automatically after setting the image threshold. The equivalent diameters were calculated from the cell-free areas. The monolayer annulus width was determined by taking the average of the annulus widths measured at four orthogonal positions. The straight-line tool of ImageJ was used for the measurements.

In the experimental study, all statistical tests were performed using the GraphPad Prism software (version 9.0.0). Two-way ANOVA with Bonferroni post hoc tests were performed to assess the effect of residual Gd³⁺ on cell viability and metabolism over time. The same statistical test was used to investigate the EGF concentration-dependent closure of the cell-free regions for both TCT and collagen I-fibronectin surfaces. A p-value<0.05 was considered statistically significant. The statistical evaluation of the microarray datasets was performed using the transcriptome analysis console (TAC) software (ThermoFisher). The raw gene expression data were normalized using the signal space transformation-robust multiarray analysis (SST-RMA) algorithm⁵⁷. A fold-change cutoff value of 2 was used to identify the differentially expressed genes⁴². For multiple comparisons in gene expression analysis, the p-value was adjusted using the false discovery rate (FDR) method⁴¹. An FDR-adjusted p-value<0.05 was considered statistically significant. All data were shown as mean±SD (standard deviation).

Magnetic field and particle trajectory simulation was performed for the three-dimensional magnetic field of the ring-cylinder magnet arrangement using the Magnetic Fields, No Currents (mfnc) interface of COMSOL Multiphysics 5.3a. A remanent flux density of 1.4 T was used to model the N52 grade neodymium magnets. The Particle Tracing for Fluid Flow (fpt) interface was used to model the cell trajectories on a 2D axisymmetric plane. The parameter values used in the simulation are provided in Table 1.

TABLE 1
Parameters used in the particle tracing simulation

| Parameter | Value | |
|---|---|---|
| Cell diameter¹ | 10 | μm |
| Cell density (assumed same as lung carcinoma | 1050 | kg m⁻³ |
| cells)² | | |
| Cell magnetic susceptibility (similar to water)³ | −9 × 10⁻⁶ | |
| Density of the medium (assumed same as water) | 1000 | kg m⁻³ |
| Dynamic viscosity of medium (assumed same as | 8.9 × 10⁻⁴ | Pa · s |
| water) | | |

¹Devalia, J. L., Sapsford, R. J., Wells, C. W., Richman, P. & Davies, R. J. Culture and comparison of human bronchial and nasal epithelial cells in vitro. Respir. Med. 84, 303-312 (1990).

### Example 1: Results and Discussion

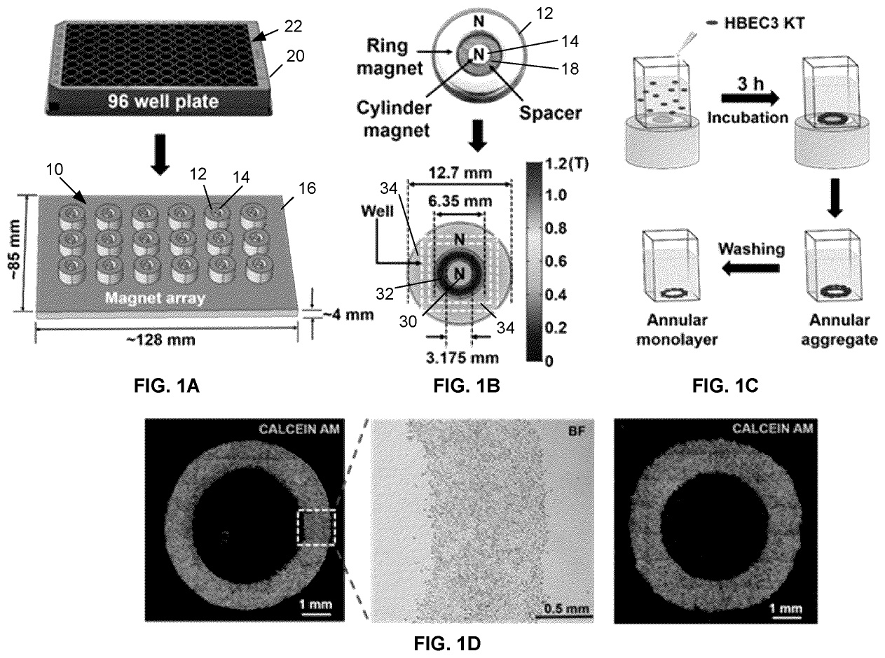

Referring now to FIGS. 1A-1D, in accordance with the teachings herein, a label-free cell aggregation method was applied to form annular HBEC3 KT monolayers using the magnetic exclusion technique. While the example is for particular types of cells, it should be noted that the method may be applied to other types of cells as well as particles as is discussed in further detail below. In this example embodiment, an array of magnets 10 were used that included an array of outer magnets 12 and an array of inner magnets 14 that are arranged on the upper surface of a substrate or support plate 16. The outer magnets 12 have a central bore. The inner magnets 14 are placed within the central bore of the outer magnets 12 such that the center of a given inner magnet 14 is located at about the center of a corresponding outer magnet 12 that the inner magnet 14 is placed within. The heights of the inner magnets 14 may be the same as the heights of the outer magnets 16. Each pair of outer and inner magnets 12 and 14 may be referred to as an outer-inner magnet pair or an outer-inner magnet arrangement.

The magnetic orientation of the outer and inner magnets 12 and 14 are the same. For instance, in this example embodiment, the upper surfaces of the outer and inner magnets 12 and 14 have a magnetic north orientation. In other embodiments, the outer and inner magnets 12 and 14 are axially magnetized and may be arranged so that their upper surfaces have a magnetic south orientation. In addition, it should be noted that the magnetic strength of the outer and inner magnets 12 and 14 may be the same or different and the magnetic field gradient is from both of the magnets 12 and 14 to the spaced region, which may be a void or include non-magnetic material. For example, the magnetic strength of the magnet depends on the type of magnet, as well as the size and shape of the magnet. For instance, the remnant magnetization of N52 magnets is ˜1.4 T and varies with the grade of the magnet. Once the magnets are selected, the magnet type is fixed. For example, the magnet type can be, but is not limited to, an N52, N42 or N35 magnet. For instance, in this example embodiment, the strength of the inner magnet is higher than the outer magnet.

The magnitude of the magnetic field gradient can vary over a wide range depending on the magnets 12 and 14 that are used. For example, the magnets can be permanent magnets with a fixed magnetic strength that is based on their size, shape and type (e.g., material, etc.). Thus, depending on the size of the spaced region, which acts as a void, the magnet size, and the size of the wells 22 the magnitude of the magnetic field can vary depending on the particular implementation.

In addition, the magnetic strength of the magnets 12 and 14, and hence the magnitude of the magnetic field gradient may be used to determine the concentration of the paramagnetic agent. For example, when the magnetic strength of the magnet pair results in a smaller magnetic field gradient, then the concentration of the paramagnetic agent in the paramagnetic medium may be increased. Alternatively, when the magnetic strength of the magnet pair results in a larger magnetic field gradient, then the concentration of the paramagnetic agent in the paramagnetic medium may be decreased. Since the paramagnetic agent may be a salt, the amount of the paramagnetic agent dissolved in the culture medium will determine its concentration.

In at least one example embodiment, an array of optional spacers 18 may also be used which have an outer wall and an inner wall with the outer wall being shaped and sized to make contact with at least several points of the inner wall of the outer magnet 12 and an inner wall to provide a central bore where the inner wall being shaped and sized to allow the inner magnet 14 to be placed therein and make contact with at least several points of the outer wall of the inner magnet 14. In cases where an approximately constant thickness for the cell aggregate is not desired, the spacers 18 do not need to be used since the spacers 18 allows the formation of an aggregate with constant width/thickness (e.g., in the radial direction). The height of the spacer 18 is shorter than the height of the inner magnet 14 so a particle structure or a cellular structure may be formed about the circumference of the inner magnet 14. This allows the bottom of a well plate 20 (described below) to sit on the top surfaces of the array of magnet pairs preferably without having a gap therebetween. The spacers 18 may be made of non-magnetic material such as plastic, for example.

In at least one embodiment, the outer magnets 12 may be ring magnets, the inner magnets 14 may be cylindrical magnets and the spacers 18 may be annular rings with an inner diameter that is slightly larger than the outer diameter of the inner magnets 14 and an outer diameter that is slightly smaller than the inner diameter of the outer magnets 12. Each pair of ring and cylinder magnets may be referred to as a ring-cylinder magnet pair or a ring-cylinder magnet arrangement. In such embodiments, the spacers 18, if used, can be annular rings.

For example, N52 grade neodymium ring magnets (12.7 mm (o.d.)×6.35 mm (i.d.)×6.35 mm) and cylinder magnets (3.175 mm (d)×6.35 mm) may be coaxially arranged in an array with an N-M orientation as shown in FIG. 1A. The parameters N and M are integers with values greater than 0 and N and M may have the same value or a different value. A well plate 20 with an array of wells 22 is placed on top of the support plate 16 over the magnet array 10. The wells 22 may have any suitable shape such as a circular, square or rectangular shape that is selected to match the desired shape of the cell aggregate that is to be formed. In this example, the well plate 20 was a 96-well flat-bottom TCT plate with square wells (7.4 mm×7.4 mm) that was placed over the magnet array 10. The center of each ring-cylinder magnet arrangement is spaced apart by ˜18 mm to align with the center of a well 22.

FIG. 1B shows the lowest magnetic field region in the annular space between the ring and cylinder magnets 12 and 14. The reproducibility of the width of the cellular aggregate strongly depends on the manufacturing tolerance of the magnet dimensions. The strength of the magnetic field in regions 30, 32, 34 and 36 is roughly about 0.4 to 0.5 T, about 0 to 0.3 T, about 0.4 to 0.5 T and about 0.5 to 0.8 T, respectively.

In general, the dimensions of the magnet pair are selected so that the inner diameter of the ring magnet falls inside a well area and doesn't interfere with any neighboring wells. As the outer diameter of the ring magnet is bigger in size than the size of a well, two adjacent wells are not used to form a cellular structure. Thus, the cellular structures may be spaced apart by at least one well.

In an alternative embodiment, instead of a well plate an individual cuvette or micro titer plate may be used to provide the wells. For example, an individual cuvette can be placed on a magnet pair compared to the magnet array for the well plate.

Cells, or particles in general, suspended in a paramagnetic solution experience a magnetic body force Fₘ that is proportional to the difference between the susceptibility of the cell X_{c} and the surrounding solution Xₛₒₗ as shown in equation 1 where²¹

$${F}_{m} = \frac{( {χ}_{c} - {χ}_{sol} ) {V}_{c}}{{μ}_{o}} ( B \cdot ∇ ) B \qquad ( 1 )$$

where χ_{c} and χₛₒₗ denote the dimensionless magnetic susceptibilities of the cell and the solution, respectively, V_{c} the cell volume, μ₀ the permeability of free space, B represents magnetic flux density, and ∇ represents the gradient operator. The magnetic susceptibilities of most cells and their culture media are typically similar. A gadolinium-based paramagnetic agent may be added to the cell culture medium to create a paramagnetic solution and induce a susceptibility difference for magnetophoresis. The magnetic susceptibility of the resulting paramagnetic solution is shown in equation 2 as:²⁹

$${χ}_{sol} = {cχ}_{mol} + {χ}_{med} \qquad ( 2 )$$

where c is the Gadolinium concentration in the cell culture medium, and χₘₒₗ and χ_{med} represent the molar susceptibility (m³ mol⁻¹) of gadolinium ions and the dimensionless susceptibility of the cell culture medium, respectively. In alternative embodiments, other transition metal salts that are paramagnetic may be used as the paramagnetic agent such as manganese sulfate monohydrate. For example, in at least one embodiment, the particles are cells and the paramagnetic agent that is prepared in a solution comprises Gadovist mixed with a phosphate—buffered saline or a cell culture media. A skilled person would be able to identify other buffers or culture media suitable for use with the embodiments described herein. However, an advantage in using Gadolinium based salt as the paramagnetic agent is that it is a chelate and will not be up taken by the cells significantly compared to other non-chelate salts which can be easily up taken by cells and may have deleterious effect.

Once the paramagnetic solution is created, the cells in the solution are allowed to incubate for a predetermined period of time to form a cellular aggregate. For instance, in this example, cells were incubated with a 25×10⁻³ M paramagnetic solution for 6 h to form compact 3D cell aggregates while maintaining high viability³⁰. Here, a 25×10⁻³ M solution of paramagnetic agent and cell culture medium is used to assemble annular HBEC3 KT monolayers within 3 h. FIG. 1C illustrates the steps involved in forming the annular HBEC3 KT aggregates.

It should be noted that length of time for the incubation period may be selected so that the cells form an aggregate that it doesn't dissociate or breaks apart when the magnet array is removed and the washing is done. Experiments can be done for the particular cell culture or particle-paramagnetic solution to determine how long the incubation period may be to avoid the breakage in the cellular or particle aggregates. The temperature and humidity that may be selected for the incubation process may be selected based on standard values that are used for particular cell culture and for when the method is applied to other particles, the temperature and humidity it is not as much of a concern.

Also, it should be noted that in general, concentration of the paramagnetic solution may be selected based on the cell viability or an effect on certain particles when this method is applied to these particles. For cells, the concentration of 25 mM does not affect cell viability²⁴.

Furthermore, it should be noted that cells are generally diamagnetic with a varying magnitude of magnetic susceptibility and size. The magnetic body force, determined from equation 1, that may be used for driving the cells into the area of lower magnetic field strength in an outer-inner magnet arrangement strongly depends on the difference between the magnetic susceptibilities of the cell and the culture medium as well as in the volume of the cells. For example, the larger the cells, and thus the larger the cell volume for a given number of cells, the faster the aggregation process is. Thus, without wishing to be bound by theory, other cell types that are diamagnetic such as adherent cell lines, non-adherent cell lines, primary cells, and stem cells will behave similarly and form cellular aggregates at the zone of low magnetic field strength. The different cell types may be co-cultured. For example, the different cell types may be epithelial cells, fibroblasts, muscle cells and other possible cell types.

The inventors further note that the above principles hold true for other particles that are not cells. Therefore, the teachings herein can be applied to other types of particles that are diamagnetic with a varying magnitude of magnetic susceptibility and sizes to create particle aggregates/particle structures for various purposes. The liquid media that is used is paramagnetic. For example, the particle aggregates may be formed to create electrodes. The particles may be various types of non-cell particles such as metal particles of a carbon-based particles. Examples of metal particles include but are not limited to copper. Examples of carbon-based particles include but are not limited to graphene oxide. The particles may be of various sizes including nanoparticles, for example.

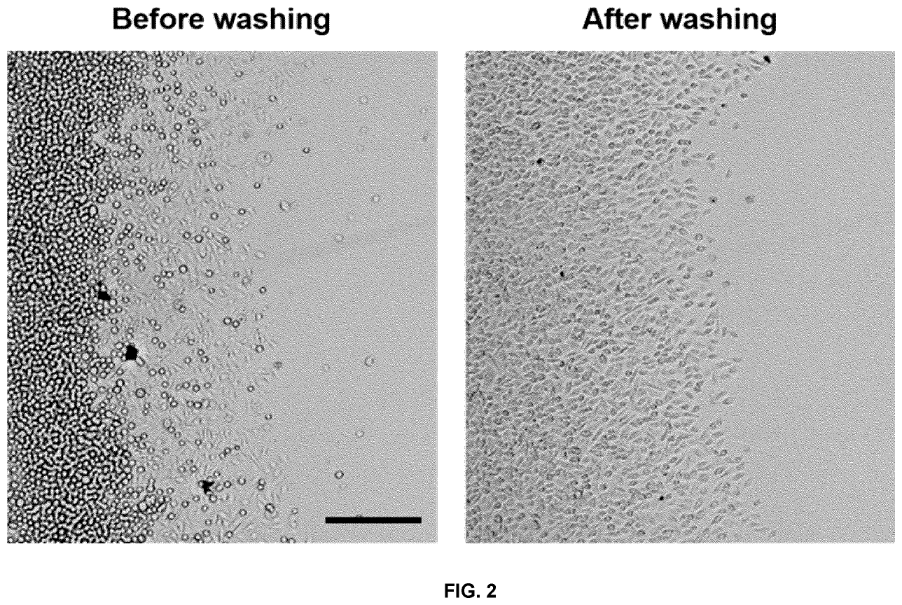

In at least one embodiment, the method may further comprise performing washing to process the cellular aggregate after incubation. For example, washing the wells after the incubation period, such as a 3 h incubation period for instance, removes loose cell aggregates, resulting in a cellular structure having a monolayer (e.g., see FIG. 2). For cells, the paramagnetic agent may affect the cell viability and so washing may be optional. However, for particles, washing is not required as there is no effect of viability with the particles. The different solutions that may be used for washing cells includes phosphate buffered saline and for particles the washing solution may be water. The concentration of the paramagnetic agent can be calculated based on the volume removed in each wash cycle. Thus, a safe concentration of the paramagnetic agent can be estimated by the number of wash cycles that are needed to remove the paramagnetic agent from the cell aggregate or particle aggregate.

The fluorescent image of an annular HBEC3 KT monolayer is shown in FIG. 1D(i). The brightfield image (the rightmost image in FIG. 1D (i)) shows the cell organization in an annular section of the monolayer. The average diameter of the cell-free region and the annulus width (e.g., outer diameter of the annular cell structure) of the HBEC3 KT monolayers are ˜3.53±0.03 mm and ˜1.08±0.05 mm, respectively (biological replicates (N)=3, technical replicates=5). The magnetic field of the cylinder magnet attracts the paramagnetic agent and displaces the cells, thereby controlling the size of the cell-free region. Therefore, for example, a cylinder magnet with a larger magnetic strength than the outer magnet can be used to displace the cells even further to make the cell-free region larger.

An ECM provides biophysical and biochemical cues which regulate cell adhesion and migration in vivo^{31,32}. Collagen type I is one of the most abundant ECM proteins in vivo and maintains tissue integrity^{33,34}. It has been used previously as a substrate for HBECs in in vitro airway models^{35,36} Fibronectin, an ECM glycoprotein, plays essential roles in various wound healing stages by modulating cell adhesion and migration^{37,38}. Hence, annular HBEC3 KT monolayers may be formed in wells in the well plate that are coated with a mixture of collagen I (500 μg mL⁻¹) and fibronectin (12 μg mL⁻¹). For example, FIG. 1D(ii) shows an annular monolayer cellular structure assembled on a coated surface after 3 h incubation. In this example, for the collagen I-fibronectin surfaces, the average diameter of the cell-free region and the annulus width of HBEC3 KT monolayers are ˜3.55±0.03 mm and ˜1.12±0.05 mm, respectively (N=3, technical replicates=5). Other natural/synthetic/decellularized ECM that may be used to coat the well plates prior to the cell assembly depending on the clinical relevance in other embodiments include, but are not limited to, collagen, elastin, laminin, fibronectin.

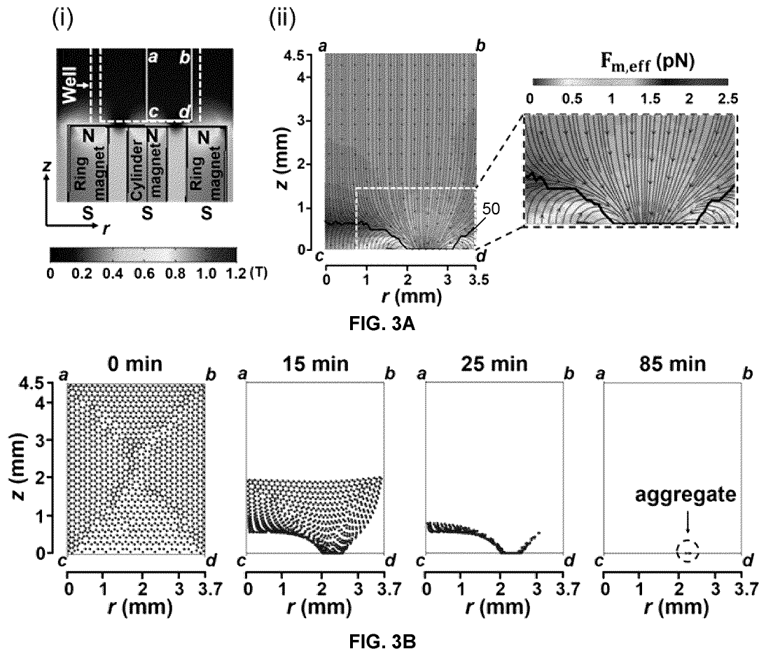

The ring-cylinder magnet axisymmetry allows one to perform a 2D particle tracing simulation using COMSOL Multiphysics 5.3a in the computational domain abcd to analyze the cell trajectories (e.g., as shown in FIG. 3A(i)). The force equation governing the motion of the cells is given by equation 3:

$${m}_{c} \frac{{dv}_{c}}{dt} = {F}_{m} + {F}_{d} + {F}_{g , net} , \qquad ( 3 )$$

where m_{c} and v_{c} represent the mass and velocity of a cell, respectively. The viscous drag F_{d} is determined by Stokes' law, i.e., F_{d}=−6πηαv_{c} where η denotes the paramagnetic solution viscosity and α the cell radius. The net gravitational force magnitude is F_{g,net}=(ρ_{c}−ρₛₒₗ)gV_{c}, where ρ_{c} denotes the cell density, ρₛₒₗ the paramagnetic solution density, and g the acceleration due to gravity. The components of Fₘ in cylindrical coordinates (r, θ, z) are

$${F}_{m , r} = \frac{( {χ}_{c} - {χ}_{sol} ) {V}_{c}}{{μ}_{o}} ( {B}_{r} \frac{\partial {B}_{τ}}{\partial r} + {B}_{Z} \frac{\partial {B}_{r}}{\partial z} ) \hat{r} , and \qquad ( 4 )$$

$${F}_{m , z} = \frac{( {χ}_{c} - {χ}_{sol} ) {V}_{c}}{{μ}_{o}} ( {B}_{r} \frac{\partial {B}_{z}}{\partial r} + {B}_{Z} \frac{\partial {B}_{z}}{\partial z} ) \hat{z} , \qquad ( 5 )$$

where \hat{r} and \hat{z} are the unit vectors. Due to axisymmetry, F_{m,θ} is zero as B_{θ} and the spatial derivatives of flux components with respect to θ vanish. The magnitude of the effective magnetophoretic force F_{m,eff} acting on a cell is \sqrt{(F_{m,z}−F_{g,net})²+(F_{m,r})²}. The magnitude of F_{m,z} is greater than F_{g,net} (˜0.26 pN) near the magnetic surfaces. Therefore, before being transported to the magnetic field minima, cells near the magnetic surfaces become levitated to a height at which F_{m,z} is balanced by F_{g,net}. The bold black curvilinear line 50 in FIG. 3A(ii) presents these levitation heights. Differences in the heights occur due to variations in the magnitude of F_{m,z} with radial distance r.

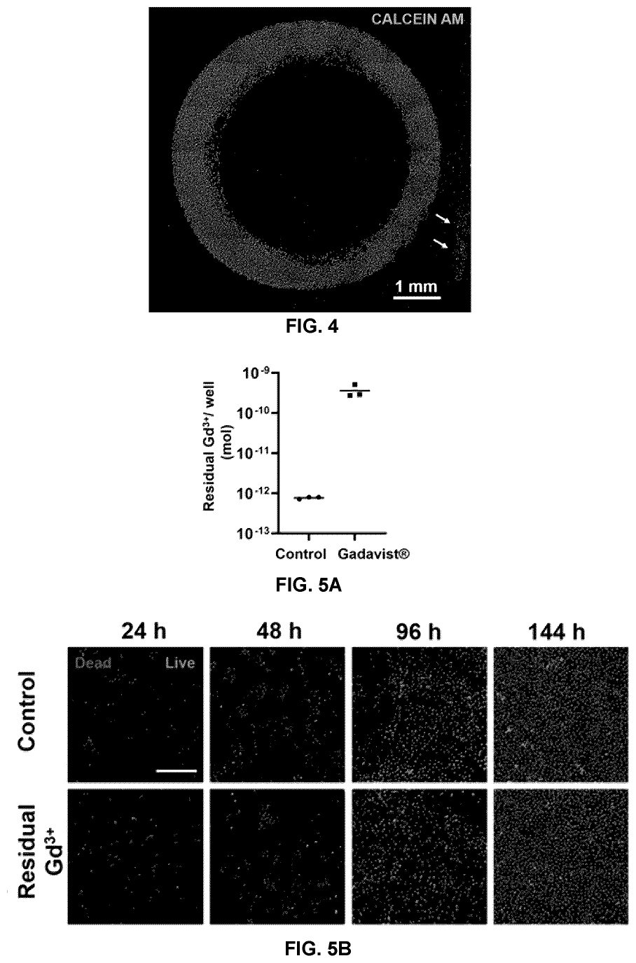

Simulated cell positions at different times are presented in FIG. 3B. Since the cells are treated as mathematical entities during the particle tracing simulation, the plots show that they overlap. The simulation confirms that the cells are transported to the magnetic field minima within 85 mins, consistent with the formation of annular monolayers on TCT surfaces as seen during experiments (e.g., see the experimental results in FIG. 4).

During experiments, at the 85-minute time point during incubation, newly formed monolayers lack well-defined boundaries and have isolated cell clusters in their vicinity. Furthermore, the short incubation time is insufficient for enabling strong cell-substrate adhesions as the monolayer integrity is disrupted during the washing step. Therefore, in general, a 3 h incubation may be more appropriate for assembling the annular monolayers. However, depending on the cell type/particle type and size, the incubation period may vary from roughly about 3 hours to 24 hours.

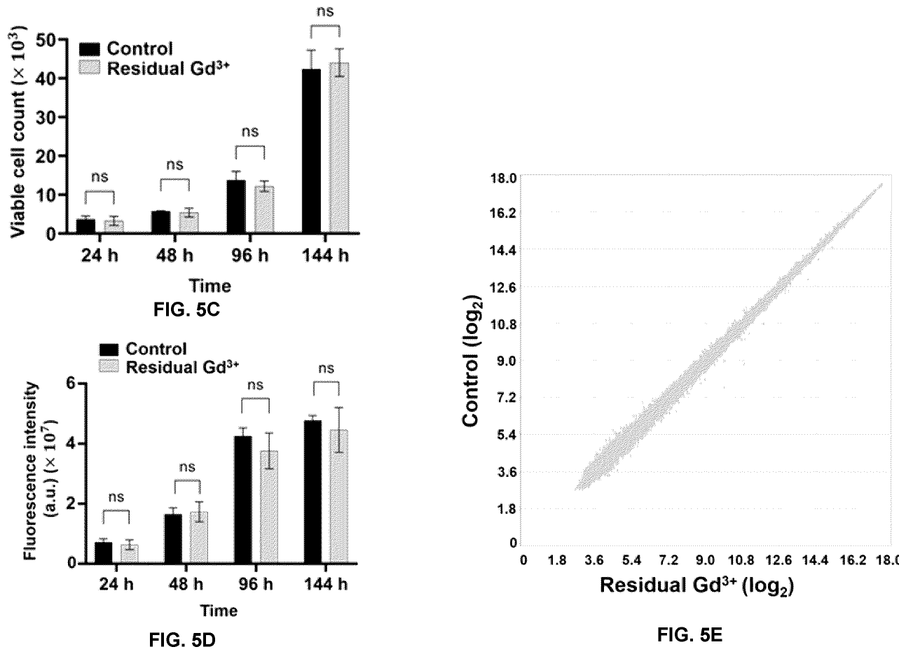

The effect of residual gadolinium ions on HBEC3 KT viability and functionality was determined during the experiments by using Inductively Coupled Plasma Mass Spectrometry (ICP-MS) to detect gadolinium ions (Gd³⁺) that remain in the wells after the washing step. The average amount of residual Gd³⁺ detected in wells containing annular monolayers was (3.58±1.32)×10⁻¹⁰ mol (N=3) (e.g., see FIG. 5A). Gd³⁺ was also detected in the control wells containing cell monolayers formed only by gravitational settling without the use of the magnetic cell free labeling, where the average amount was (7.68±0.47)×10⁻¹³ mol (N=3). The detection of Gd³⁺ in the control wells is possibly due to spectroscopic interference caused by selenium³⁹. Selenium was present in the cell culture media as an essential nutrient for cell proliferation which is incorporated into various proteins⁴⁰.

The effect of the residual Gd³⁺ on cell viability was assessed at 24, 48, 96, and 144 h using a live/dead assay. FIG. 5B shows the fluorescent images of live (blue) and dead (green) cells at different times. The numbers of viable HBEC3 KT cells were quantified from the fluorescent images using ImageJ (as shown in FIG. 5C). FIGS. 5B and 5C show that the viable cell counts increase as cells proliferate over time, and the effect of residual Gd³⁺ on cell viability is insignificant. Cell metabolism, which is an indicator of viability, was also measured using a resazurin-based assay (Presto Blue HS). The fluorescence intensity values, which are proportional to the cell metabolic activity, are shown in FIG. 5D. The increase in the fluorescence intensity values over time indicates cell proliferation. No significant difference was observed between the cell metabolism of the control and the Gadolinium groups.

To investigate the effect of residual Gd³⁺ on the HBEC3 KT gene expression profiles, microarray gene expression analysis was performed (Clariom S assay, ThermoFisher) to evaluate the differential expression of 21,448 genes. For multiple comparisons in the statistical analysis, the p-value was adjusted using the false discovery rate (FDR) method⁴¹. A combination of FDR-adjusted p-value and fold-change criteria was used to identify the differentially expressed genes⁴². A comparison of the expression data of control and Gadolinium groups is presented in FIG. 5E which shows that there are no statistically significant differentially expressed genes. This result indicates that the impact of residual Gd³⁺ on the HBEC3 KT transcriptional profiles is insignificant.

The cell-free area closure of the annular monolayers was also studied in the experiments. EGF promotes the proliferation and migration of epithelial cells during wound healing^{43,44}. The ability of EGF to enhance wound closure has previously been demonstrated with scratch assays^{45,46}. HBEC3 KT annular monolayers were incubated with 0, 0.16, and 0.8 ng mL⁻¹ EGF to demonstrate the EGF concentration-dependent closure of the cell-free regions in the magnetically assembled structures. The maximum EGF concentration selected was 0.8 ng mL⁻¹, which is the concentration used to culture HBEC3 KT cells. The annular monolayers were produced on TCT and collagen I-fibronectin coated well surfaces to investigate the influence of the underlying substrate on the closure rate.

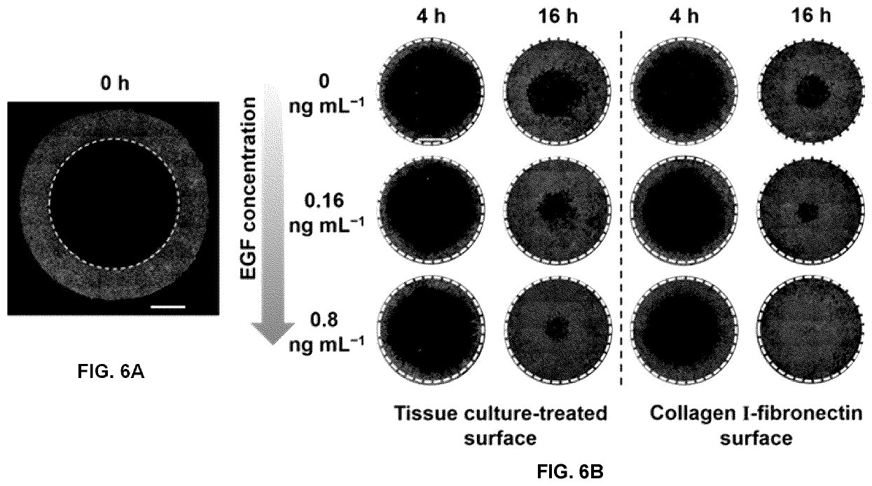

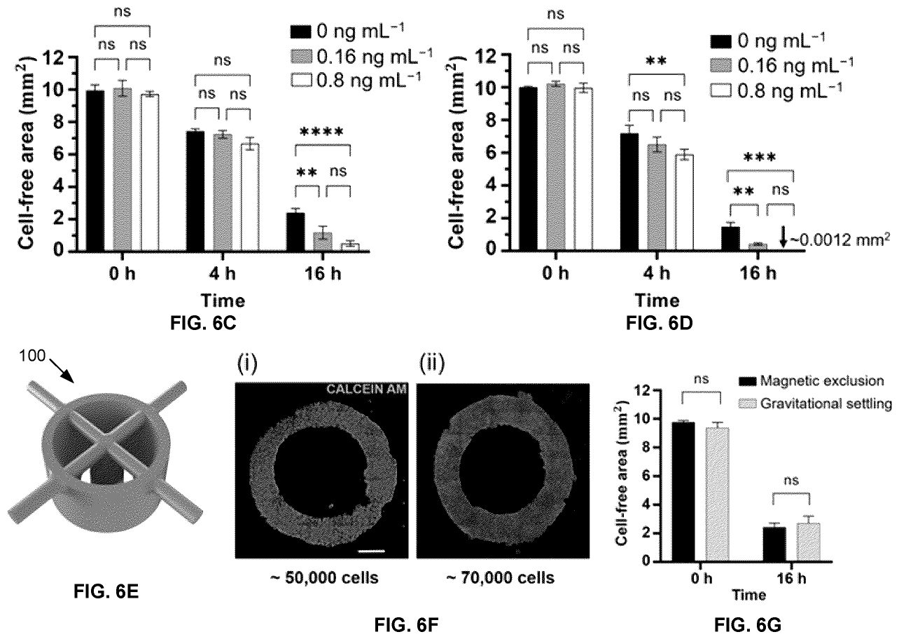

The initial boundary of the cell-free region is marked by a white dotted line (e.g., see FIG. 6A). FIG. 6B shows fluorescent images of the cell-free regions at 4 and 16 h for different EGF concentrations and surface conditions. The cell-free areas on both TCT (FIG. 6C) and collagen I-fibronectin surfaces (FIG. 6D) were measured at 0, 4, and 16 h for all EGF concentrations using ImageJ. The EGF concentration-dependent closures of the cell-free regions on TCT and collagen I-fibronectin surfaces differ at 16 h. In contrast to TCT surfaces, there is a significant decrease in the cell-free areas on collagen I-fibronectin surfaces at 4 h for 0.8 ng mL⁻¹ EGF, with a near-complete closure at 16 h.

The closure rates are calculated by measuring the differences between the mean initial and final cell-free areas at 0 h and 16 h. The closure rates for TCT surfaces are ˜0.47±0.03 mm² h⁻¹ (0 ng mL⁻¹), ˜0.56±0.05 mm² h⁻¹ (0.16 ng mL⁻¹), and ˜0.58±0.01 mm² h⁻¹ (0.8 ng mL⁻¹) whereas for collagen I-fibronectin surfaces, the rates are ˜0.53±0.02 mm² h⁻¹ (0 ng mL⁻¹), ˜0.61±0.01 mm² h⁻¹ (0.16 ng mL⁻¹), and ˜0.62±0.02 mm² h⁻¹ (0.8 ng mL⁻¹) (N=3).

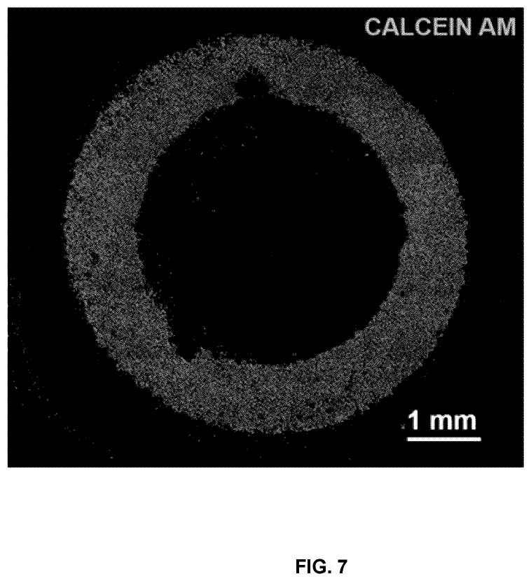

Next, control experiments were performed to investigate the effect of residual Gadolinium on cell-free area closure. 3D-printed annular inserts were used to produce cell monolayers by gravitational settling (e.g., see FIG. 6E). The gravity-assisted annular monolayers formed on TCT surfaces after 3 h were not as compact as the magnetically assembled monolayers (e.g., see FIG. 7). Since the 3 h incubation time was insufficient for the cells settling under gravity to adhere to the surface firmly, the incubation time was increased to 9 h to enhance cell attachment.

FIG. 6F(i) shows a fluorescent image of an example of a gravity-assisted annular monolayer after a 9 h incubation. Although the monolayers were compact, the annular sections had inconsistent widths. Increasing cell seeding from ˜50,000 to ˜70,000 cells resulted in more consistent annulus widths (FIG. 6F(ii) shows an example). The average diameters of the cell-free regions and annulus widths for the gravity-assisted HBEC3 KT monolayers (cell seeding=˜70,000) were ˜3.45±0.07 mm and ˜1.18±0.09 mm, respectively (N=1, technical replicates=5). To assess cell-free area closure, gravity-assisted monolayers were incubated without introducing EGF for 16 h. No significant difference was observed between the cell-free areas of the gravity-assisted and magnetically assembled annular monolayers, respectively, at 0 and 16 h, as shown in FIG. 6G. Thus, the effect of residual Gadolinium on the closure of magnetically assembled monolayers was determined to be insignificant.

Numerical model: The Fisher—Kolmogorov—Petrovsky—Piskunov (FKPP) reaction-diffusion equation is routinely used to model the closure of rectangular cell-free areas in scratch-based assays^{27,47,48}. The FKPP equation consists of a diffusion term that describes cell migration and a source term representing cell proliferation. The one-dimensional axisymmetric form of the FKPP equation is given by equation (6):⁴⁹

$$\frac{\partial U}{\partial t} = \frac{1}{r} \frac{\partial }{\partial r} ( rD \frac{\partial U}{\partial r} ) + GU , \qquad ( 6 )$$

where U denotes the cell density (cells mm⁻²) and D the cell diffusivity (mm² h⁻¹). The source term consists of a logistic growth function G=λ(1−U/K) with growth rate λ(h⁻¹) and carrying capacity K (cells mm⁻²)⁴⁷. The carrying capacity is the maximum density for a cell monolayer when contact inhibition reduces the net cell proliferation to zero⁴⁷. The classical FKPP model assumes a constant cell diffusivity D=D₀, where D₀ is the diffusivity for isolated cells^{27,47}. In contrast, the Porous-Fisher model, which is an extension of the FKPP model, uses a diffusivity function D=D₀(U/K)^{m} that increases with cell density (m>0), where D₀ denotes the maximum diffusivity^{47,50}. While some investigators have reported better fits of scratch assay experimental data with the Porous-Fisher model, others have found that model selection depends on the cell type^{28,47}.

Besides cell migration, cell proliferation leads to the closure of cell-free areas. To determine Δ, a logistic growth equation⁵¹ was used as shown in equations 7 and 8:

$$\frac{dU}{dt} = λ U ( 1 - \frac{U}{K} ) , i . e . , \qquad ( 7 )$$

$$U = \frac{{KU}_{o}}{{U}_{o} + ( K - {U}_{o} ) {e}^{- λ t}} , \qquad ( 8 )$$

where U₀ denotes the initial cell density. The maximum cell density determined by counting cells in the confluent regions of annular HBEC 3KT monolayers gives K=1690 cells mm⁻², which is consistent with the previously reported values^{28,52}. Experimentally derived values of U at 16 h and U₀ were used to calculate λ from Equation 8. For 0 ng mL⁻¹ EGF, λ˜0.01 h⁻¹, whereas λ˜0.03 h⁻¹ for both 0.16 and 0.8 ng mL⁻¹ EGF.

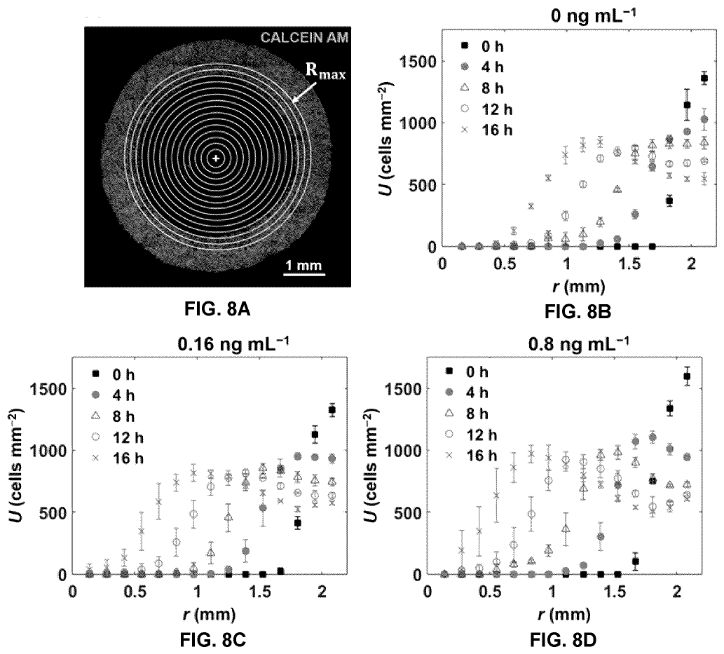

Brightfield images of the annular monolayers were taken every 2 h over 16 h to track the closures of the cell-free areas on TCT surfaces. Consistent with the monolayer geometry, circular tracks of ˜0.14 mm width were superimposed on the brightfield images to manually count the cells within the domain 0≤r≤Rₘₐₓ. For illustration, these circular tracks were superimposed on a fluorescent image as shown in FIG. 8A. The total number of cells counted in each track was divided by the respective annulus area to generate experimental cell density datasets. In the cell density plots, each track was represented by the radial distance of its midpoint from the center of the annular monolayer. The experimental cell density profiles for different EGF concentrations are shown in FIGS. 8B, 8C, and 8D.

The finite volume method (FVM) with a time marching scheme was used to discretize and predict cell density (Uₚ) values from Equation 6. A linear combination of the classical FKPP and Porous-Fisher diffusivity functions were used, i.e., D=D₀+D₁(U/K)^{m}, to model the spatiotemporal change in cell density on the TCT surfaces. The combined diffusivity function was previously used to fit cell density profiles of scratch assays with rectangular cell-free areas⁵⁰. A zero net flux boundary condition was imposed at both r=0 and r=Rₘₐₓ^{28,50}.

FIGS. 8B, 8C, and 8D shows that the cell density at r=Rₘₐₓ decreases over time as the cell front traverses the cell-free area. As the cell density decreases at the boundary Rₘₐₓ, the zero net flux condition applied at this boundary is an approximation. A Genetic Algorithm (GA)-based error minimization approach was implemented to minimize the root mean squared error (RMSE) between Uₚ and the experimental (Uₑₓₚ) cell density values⁵³ as shown in equation 9.

$$R M S E = \sqrt{\frac{{\sum }_{i = 1}^{15} {\sum }_{j = 1}^{4} {( {U}_{P} ( i , j ) - {U}_{exp} ( i , j ) )}^{2}}{15 \times 4}} \qquad ( 9 )$$

The cell density was measured at 15 locations within 0≤r≤Rₘₐₓ and the index i denotes the location number. The index j indicates timepoints, where j=1, 2, 3 and 4 corresponds to t=4, 8, 12 and 16 h. The reaction-diffusion equation was solved for different EGF concentrations using constraints on the unknown variables D₀, D₁ and m. The limits for D₀ are 0≤D₀≤0.004 mm² h⁻¹, where D₀=0.004 mm² h⁻¹ is the maximum isolated-cell diffusivity value reported in the literature^{28,50}.

The experimentally obtained cell-free area closure rates were approximately two orders higher than the maximum value of D₀(Section 2.3). Hence, the upper limit of D₁ is increased by an order higher than the experimental closure rates for optimization within 0≤D₁≤4 mm² h⁻¹. The exponent m determines whether the diffusivity D changes linearly or non-linearly with a change in cell density. However, the biological meaning of m is not yet fully understood⁴⁷. Here, the limits 0≤m≤4 are considered, where m=4 is the highest value reported in the literature^{28,50}. The experimentally derived values of λ are used in the model.

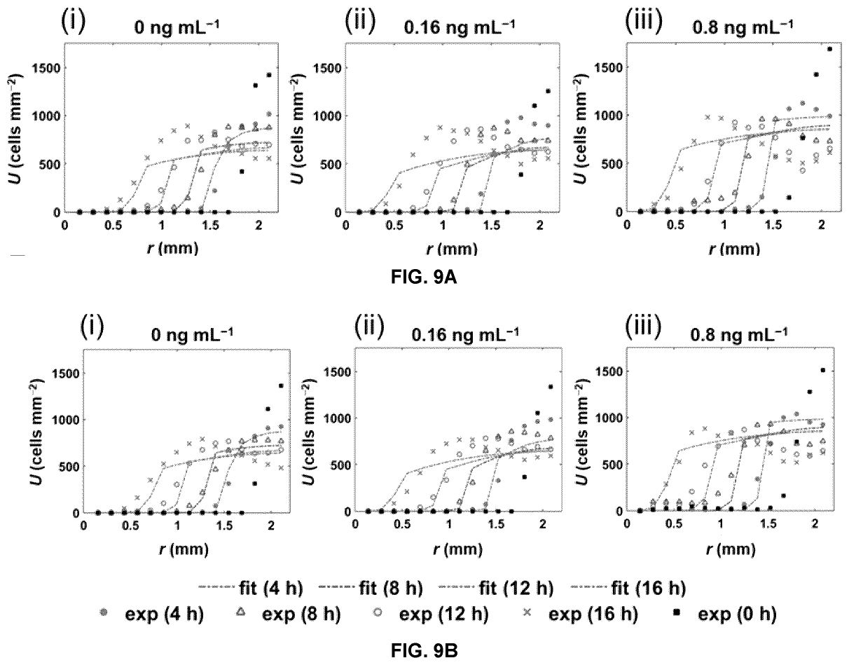

The numerical solutions provide a lower value of D₀ for 0.16 ng mL⁻¹ and 0.8 ng mL⁻¹ than for 0 ng mL⁻¹ EGF. Since EGF stimulates cell migration, higher D₀ values for 0.16 ng mL⁻¹ and 0.8 ng mL⁻¹ are expected^{45,46}. Therefore, for these concentrations, the lower limit of D₀ in 0≤D₀≤0.004 mm² h⁻¹ was revised to D₀|_{EGF=0}, where D₀|_{EGF=0} is the optimal D₀ value obtained for 0 ng mL⁻¹. For model cross-validation, the values of D₀, D₁ and m obtained from one replicate (Replicate 1) were used to fit the experimental data of the other replicate (Replicate 2). The optimal values of D₀, D₁ and m for Replicate 1, and the RMSE values for Replicates 1 and 2 are given in Table 2. The predicted cell density profiles for Replicates 1 and 2 are shown in FIGS. 9A and 9B.

A dimensionless number

$$φ = \frac{{t}_{max} \sqrt{{D}_{1} λ}}{{R}_{max}}$$

was used to compare the numerical results for different EGF concentrations. This number accounted for both cell migration and proliferation, where a higher φ indicates faster closure. The parameter Rₘₐₓ depends on the dimensions of the cylinder magnet and tₘₐₓ denotes the time for complete closure of the cell-free region. The parameters tₘₐₓ and Rₘₐₓ are constants for a given cell type and magnet dimensions and hence, φ is directly proportional to \sqrt{D₁λ}. Here, tₘₐₓ=24 h was selected, considering that cell-free area closure was ˜75% (0 ng mL⁻¹)-˜95% (0.8 ng mL⁻¹) at 16 h. Since D₀<D₁, only the diffusivity D₁ was used in the expression of φ. The values of φ for different EGF concentrations are provided in Table 2.

TABLE 2
Parameter values obtained from the numerical
solutions of the reaction-diffusion equation

| EGF | D₀ | D₁ | | λ | RMSE | RMSE | |
|---|---|---|---|---|---|---|---|
| (ng | (mm² h⁻¹) | (mm² | | (h⁻¹) | (Repli- | (Repli- | |
| mL⁻¹) | (×10⁻³) | h⁻¹) | m | (×10⁻²) | cate 1) | cate 2) | φ |
| 0.00 | 1.94 | 3.02 | 1.97 | 1.25 | 111.73 | 86.05 | 2.12 |
| 0.16 | 3.06 | 3.02 | 1.97 | 2.91 | 142.15 | 116.21 | 3.23 |
| 0.80 | 3.91 | 3.75 | 2.62 | 2.91 | 154.45 | 142.09 | 3.60 |

The present teachings describe a rapid, label-free magnetic exclusion technique to create cell-free areas which may be used for a variety of purposes, including but not limited to, studying cell migration. The magnetic field distribution of the ring-cylinder magnet arrangement controls the cell aggregate shape and size. When paramagnetic suspensions of HBEC3 KT cells were exposed to the magnetic field, the magnetic force Fₘ drives the paramagnetic agent towards the region of the highest magnetic field strength on the well surface, displacing the cells towards the region of lowest field strength. As proof of concept, HBEC3 KT cells suspended in 25×10⁻³ M paramagnetic salt solution were assembled into annular monolayers on both TCT and collagen I-fibronectin surfaces within 3 h. In contrast, traditional cell migration assays involving physical scratches or physical inserts require 20-48 h for the cells to be confluent^{11,17}. Thus, the magnetic exclusion technique, according to the teachings herein, leads to a ˜10-fold decrease in the overall assay time.

The ICP-MS results show retention of Gd³⁺ in the wells. Gd³⁺ retention was possibly due to cellular uptake or residual Gadolinium solution remaining in the wells after the washing step. The cellular uptake of Gd³⁺ can potentially be detected using techniques such as in vivo cellular MRI⁵⁴. Live/dead and metabolism assays indicated that the residual Gd³⁺ is not toxic to the cells. Moreover, the microarray results showed that the effect of residual Gd³⁺ on the HBEC3 KT transcriptional profiles is statistically insignificant and doesn't affect the functionality of the cells.

The cell-free area closure study indicates that collagen I-fibronectin coating enhances the closure rates (FIG. 6B). The rapid closure rates for collagen I-fibronectin surfaces were likely due to the binding of these ECM proteins with integrins (transmembrane receptors) that facilitate cell migration^{55,56}. Formation of consistent and highly reproducible scratch is critical for any cell migration study to reduce the variability in the sample. The reported work demonstrates the formation of wounds of consistent size and thus improving the outcome. The numerical model for cell-free area closure tracks the moving front of the experimental cell density profiles for all EGF concentrations. The φ values for 0.16 and 0.8 ng mL⁻¹ EGF are higher than that for 0 ng mL⁻¹ (Table 2). These numerical results are consistent with the experimental observations revealing that higher EGF concentrations have faster closure rates. However, the model cannot predict the decrease in cell density at r˜2 mm and the increase at r˜1 mm. This spatial variation in cell density is possibly due to the combined effect of the annular geometry and the rapid closure of the cell-free area. Complex functional forms of D and G are required to precisely capture the spatiotemporal change in cell density⁵⁰.

The magnetic exclusion technique is rapid, easy to use, and can readily be integrated into existing microwell plates without any modifications to the microwell plates. This label-free, scaffold-free technique can easily be adapted to form annular aggregates for various purposes such as, but not limited to, 3D cell invasion assays, for example. The magnetic exclusion technique may also be used for preclinical applications such as the fabrication of smooth muscle rings for asthma and inflammatory bowel disease models. This may be possible by layering different cell types and monitoring the response of different environmental factors to the cellular aggregate. For instance, for asthma smooth muscle cell, fibroblasts and epithelial cells can be layered together.

In another aspect, the method according to the teachings herein may be used for generating cellular aggregates for use in cell migration assays, 3D cell invasion assays, wound healing assays, study cell interactions, drug discovery and drug screening, and/or disease models, where the disease models include asthma models and/or Inflammatory Bowel Disease models.

For example, the method may comprise generating cellular aggregates an ECM coated surface in accordance with the teachings herein and the invasion of cells into the ECM can be studied for invasion assays.

As another example, cellular aggregates of different cell types (either layer on layer or two adjacent cell aggregates) can be formed and the cell-cell interactions can be studied. To form layer on layer cellular aggregates with two different cell types, the first cell type is suspended in the paramagnetic culture medium and added to the microwell plates. When the well plate is placed on the magnet array, the cellular aggregate is formed. Following a washing step, the second cell type is suspended to the paramagnetic medium and added to the same wells. The cellular aggregate of the second cell type is thus formed on the first cell type cellular aggregate. The different layers may be of different shapes and sizes depending on the magnet array. In an alternative embodiment, prior to adding the second cell type with the paramagnetic medium to the same wells, a given structure can be added first if it is desired for the layers to be separated by the given structure. For example, a natural ECM or a synthetic ECM may be added to the wells after the first washing step and before the cell culture with the second cell type is added to the wells so that at least one pair of successive layers have an ECM between them.

As another example, the method may comprise generating cellular aggregates in accordance with the teachings herein and using the cellular aggregates for the fabrication of smooth muscle rings for use in Asthma models or Inflammatory Bowel Disease models.

As another example, the method may comprise generating cellular aggregates in accordance with the teachings herein and using the cellular aggregates to study cell migration and/or wound healing as was described in the experimental study.

In another aspect, there is provided at least one embodiment of a kit that includes a paramagnetic agent, and an array of magnet pairs for applying an external magnetic field gradient to at least one receptacle. The magnet pairs include an outer magnet and an inner magnet, which may also be referred to as an external magnet and an internal magnet, that is disposed within the outer magnet with a spaced region therebetween as was discussed previously with respect to FIGS. 1A-1C. In at least one embodiment, the kit may include spacers that are shaped for placement within the magnet pairs. In at least one embodiment, the kit further may further include a cell culture plate, a cuvette or a micro titer plate that provides the at least one receptacle.

While the applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without generally departing from the embodiments described herein. For example, while the teachings described and shown herein may comprise certain elements/components and steps, modifications may be made as is known to those skilled in the art. For example, selected features from one or more of the example embodiments described herein in accordance with the teachings herein may be combined to create alternative embodiments that are not explicitly described. All values and sub-ranges within disclosed ranges are also disclosed. The subject matter described herein intends to cover and embrace all suitable changes in technology. Accordingly, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### Full Citations for Documents Referred to in the Application

1. Trepat, X., Chen, Z. & Jacobson, K. Cell Migration. in Comprehensive Physiology 176, 100-106 (John Wiley & Sons, Inc., 2012).
2. Qu, F., Guilak, F. & Mauck, R. L. Cell migration: implications for repair and regeneration in joint disease. Nat. Rev. Rheumatol. 15, 167-179 (2019).
3. Devreotes, P. & Horwitz, A. R. Signaling networks that regulate cell migration. Cold Spring Harb. Perspect. Biol. 7, (2015).
4. Vicente-Manzanares, M., Webb, D. J. & Horwitz, A. R. Cell migration at a glance. J. Cell Sci. 118, 4917-4919 (2005).
5. de Oliveira, S., Rosowski, E. E. & Huttenlocher, A. Neutrophil migration in infection and wound repair: going forward in reverse. Nat. Rev. Immunol. 16, 378-391 (2016).
6. Yarrow, J. C., Perlman, Z. E., Westwood, N. J. & Mitchison, T. J. A high-throughput cell migration assay using scratch wound healing, a comparison of image-based readout methods. BMC Biotechnol. 4, 1-9 (2004).
7. Fenteany, G., Janmey, P. A. & Stossel, T. P. Signaling pathways and cell mechanics involved in wound closure by epithelial cell sheets. Curr. Biol. 10, 831-838 (2000).
8. Etienne-Manneville, S. & Hall, A. Integrin-mediated activation of Cdc42 controls cell polarity in migrating astrocytes through PKCζ. Cell 106, 489-498 (2001).
9. Magdalena, J., Millard, T. H. & Machesky, L. M. Microtubule involvement in NIH 3T3 Golgi and MTOC polarity establishment. J. Cell Sci. 116, 743-756 (2003).
10. Hulkower, K. I. & Herber, R. L. Cell migration and invasion assays as tools for drug discovery. Pharmaceutics 3, 107-124 (2011).
11. Liang, C. C., Park, A. Y. & Guan, J. L. In vitro scratch assay: A convenient and inexpensive method for analysis of cell migration in vitro. Nat. Protoc. 2, 329-333 (2007).
12. Kramer, N. et al. In vitro cell migration and invasion assays. Mutat. Res. Mutat. Res. 752, 10-24 (2013).
13. Riahi, R., Yang, Y., Zhang, D. D. & Wong, P. K. Advances in wound-healing assays for probing collective cell migration. J. Lab. Autom. 17, 59-65 (2012).
14. Keese, C. R., Wegener, J., Walker, S. R. & Giaever, I. Electrical wound-healing assay for cells in vitro. Proc. Natl. Acad. Sci. U.S.A 101, 1554-1559 (2004).
15. Poujade, M. et al. Collective migration of an epithelial monolayer in response to a model wound. Proc. Natl. Acad. Sci. U.S.A 104, 15988-15993 (2007).
16. Jonkman, J. E. N. et al. An introduction to the wound healing assay using live-cell microscopy. Cell Adhes. Migr. 8, 440-451 (2014).
17. Gough, W. et al. A quantitative, facile, and high-throughput image-based cell migration method is a robust alternative to the scratch assay. J. Biomol. Screen. 16, 155-163 (2011).
18. Timm, D. M. et al. A high-throughput three-dimensional cell migration assay for toxicity screening with mobile device-based macroscopic image analysis. Sci. Rep. 3, 1-8 (2013).
19. Souza, G. R. et al. Three-dimensional tissue culture based on magnetic cell levitation. Nat. Nanotechnol. 5, 291-296 (2010).
20. Patil, U. S. et al. In vitro/in vivo toxicity evaluation and quantification of iron oxide nanoparticles. International Journal of Molecular Sciences 16, (2015).
21. Akiyama, Y. & Morishima, K. Label-free cell aggregate formation based on the magneto-Archimedes effect. Appl. Phys. Lett. 98, (2011).
22. Anil-Inevi, M. et al. Biofabrication of in situ Self Assembled 3D Cell Cultures in a Weightlessness Environment Generated using Magnetic Levitation. Sci. Rep. 8, 1-10 (2018).
23. Abdel Fattah, A. R. et al. In Situ 3D Label-Free Contactless Bioprinting of Cells through Diamagnetophoresis. ACS Biomater. Sci. Eng. 2, 2133-2138 (2016).
24. Mishriki, S. et al. Rapid Magnetic 3D Printing of Cellular Structures with MCF-7 Cell Inks. Research 2019, 1-13 (2019).
25. Tocchio, A. et al. Magnetically Guided Self-Assembly and Coding of 3D Living Architectures. Adv. Mater. 30, 1-8 (2018).
26. Gupta, T. et al. Label-Free Magnetic-Field-Assisted Assembly of Layer-on-Layer Cellular Structures. ACS Biomater. Sci. Eng. 6, 4294-4303 (2020).
27. Tremel, A. et al. Cell migration and proliferation during monolayer formation and wound healing. Chem. Eng. Sci. 64, 247-253 (2009).
28. Jin, W. et al. Reproducibility of scratch assays is affected by the initial degree of confluence: Experiments, modelling and model selection. J. Theor. Biol. 390, 136-145 (2016).
29. Coey, J. M. D., Aogaki, R., Byrne, F. & Stamenov, P. Magnetic stabilization and vorticity in submillimeter paramagnetic liquid tubes. Proc. Natl. Acad. Sci. U.S.A 106, 8811-8817 (2009).
30. Abdel Fattah, A. R. et al. 3D cellular structures and co-cultures formed through the contactless magnetic manipulation of cells on adherent surfaces. Biomater. Sci. 6, 683-694 (2018).
31. Wang, W. Y. et al. Extracellular matrix alignment dictates the organization of focal adhesions and directs uniaxial cell migration. APL Bioeng. 2, 1-16 (2018).
32. Gkretsi, V. & Stylianopoulos, T. Cell adhesion and matrix stiffness: Coordinating cancer cell invasion and metastasis. Front. Oncol. 8, (2018).
33. Xue, M. & Jackson, C. J. Extracellular Matrix Reorganization During Wound Healing and Its Impact on Abnormal Scarring. Adv. Wound Care 4, 119-136 (2015).
34. Booth, A. J. et al. Acellular normal and fibrotic human lung matrices as a culture system for in vitro investigation. Am. J. Respir. Crit. Care Med. 186, 866-876 (2012).
35. Ishikawa, S., Ishimori, K. & Ito, S. A 3D epithelial-mesenchymal co-culture model of human bronchial tissue recapitulates multiple features of airway tissue remodeling by TGF-β1 treatment. Respir. Res. 18, 1-11 (2017).
36. Vaughan, M. B., Ramirez, R. D., Wright, W. E., Minna, J. D. & Shay, J. W. A three-dimensional model of differentiation of immortalized human bronchial epithelial cells. Differentiation 74, 141-148 (2006).
37. Patten, J. & Wang, K. Fibronectin in development and wound healing. Adv. Drug Deliv. Rev. 170, 353-368 (2021).
38. Hsiao, C. Te et al. Fibronectin in cell adhesion and migration via N-glycosylation. Oncotarget 8, 70653-70668 (2017).
39. Wilschefski, S. C. & Baxter, M. R. Inductively Coupled Plasma Mass Spectrometry: Introduction to Analytical Aspects. Clin. Biochem. Rev. 40, 115-133 (2019).
40. Huawei, Z. Selenium as an essential micronutrient: Roles in cell cycle and apoptosis. Molecules 14, 1263-1278 (2009).
41. Jafari, M. & Ansari-Pour, N. Why, when and how to adjust your P values?Cell J. 20, 604-607 (2019).
42. Zhao, B., Erwin, A. & Xue, B. How many differentially expressed genes: A perspective from the comparison of genotypic and phenotypic distances. Genomics 110, 67-73 (2018).
43. Hardwicke, J., Schmaljohann, D., Boyce, D. & Thomas, D. Epidermal growth factor therapy and wound healing—Past, present and future perspectives. Surgeon 6, 172-177 (2008).
44. Bodnar, R. J. Epidermal Growth Factor and Epidermal Growth Factor Receptor: The Yin and Yang in the Treatment of Cutaneous Wounds and Cancer. Adv. Wound Care 2, 24-29 (2013).
45. PUDDICOMBE, S. M. Involvement of the epidermal growth factor receptor in epithelial repair in asthma. FASEB J. 14, 1362-1374 (2000).
46. Kim, J. S., McKinnis, V. S., Nawrocki, A. & White, S. R. Stimulation of migration and wound repair of guinea-pig airway epithelial cells in response to epidermal growth factor. Am. J. Respir. Cell Mol. Biol. 18, 66-74 (1998).
47. Warne, D. J., Baker, R. E. & Simpson, M. J. Using Experimental Data and Information Criteria to Guide Model Selection for Reaction-Diffusion Problems in Mathematical Biology. Bull. Math. Biol. 81, 1760-1804 (2019).
48. Maini, P. K., McElwain, D. L. S. & Leavesley, D. I. Traveling Wave Model to Interpret a Wound-Healing Cell Migration Assay for Human Peritoneal Mesothelial Cells. Tissue Eng. 10, 475-482 (2004).
49. McCue, S. W. et al. Hole-closing model reveals exponents for nonlinear degenerate diffusivity functions in cell biology. Phys. D Nonlinear Phenom. 398, 130-140 (2019).
50. Lagergren, J. H., Nardini, J. T., Baker, R. E., Simpson, M. J. & Flores, K. B. Biologically-informed neural networks guide mechanistic modeling from sparse experimental data. PLoS Comput. Biol. 16, 1-29 (2020).
51. Charlebois, D. A. & Balezsi, G. Modeling cell population dynamics. In Silico Biol. 13, 21-39 (2019).
52. Jin, W., Lo, K. Y., Chou, S. E., McCue, S. W. & Simpson, M. J. The role of initial geometry in experimental models of wound closing. Chem. Eng. Sci. 179, 221-226 (2018).
53. Deb, K., Pratap, A., Agarwal, S. & Meyarivan, T. A fast and elitist multiobjective genetic algorithm: NSGA-II. IEEE Trans. Evol. Comput. 6, 182-197 (2002).
54. Liu, W. & Frank, J. A. Detection and quantification of magnetically labeled cells by cellular MRI. Eur. J. Radiol. 70, 258-264 (2009).
55. Huttenlocher, A. & Horwitz, A. R. Integrins in cell migration. Cold Spring Harb. Perspect. Biol. 3, 1-16 (2011).
56. Palecek, S. P., Loftus, J. C., Ginsberg, M. H., Lauffenburger, D. A. & Horwitz, A. F. Integrin-ligand binding properties govern cell migration speed through cell-substratum adhesiveness [published erratum appears in Nature 1997 Jul. 10; 388(6638):210]. Nature 385, 537-540 (1997).
57. Irizarry, R. A. et al. Summaries of Affymetrix GeneChip probe level data. Nucleic Acids Res. 31, e15 (2003).

1. A method of generating cell-free regions in a cellular aggregate using label-free magnetic manipulation, wherein the method comprises:
suspending cells in a paramagnetic culture medium to create a magnetic susceptibility difference, the cells being diamagnetic;
seeding the cells and the paramagnetic culture medium into microwells; and
placing the microwells over an array of magnet pairs where each magnet pair creates a magnetic field gradient,
wherein the magnetic susceptibility difference and the magnetic field gradient drive the cells towards a spaced region between magnets in the magnet pair having a lowest magnetic field strength to create the cellular aggregate with a cell-free area where the cellular aggregate surrounds the cell free-area.

2. The method of claim 1, wherein each magnet pair includes an outer magnet and an inner magnet and the magnetic field gradient is from both each magnet to the spaded region.

3. The method of claim 2, wherein the inner magnet is approximately coaxially aligned with the outer magnet using a spacer.

4. (canceled)

5. The method of claim 2, wherein the outer magnet comprises a ring magnet and the inner magnet comprises a cylinder magnet.

6. The method of claim 2, wherein an internal wall of the outer magnet and outer wall of the inner magnet is selected to have a desired shape to generate the cellular aggregate where the cell-free area has the desired shape.

7. The method of claim 6, wherein the desired shape includes a rectangular shape, a square shape, an elliptical shape or a circular shape.

8. The method of claim 1, wherein the array of magnet pairs is disposed on a support plate.

9. The method of claim 1, wherein the microwells are provided in a well plate, a cell culture plate, a micro titer plate or an individual cuvette.

10. The method of claim 1, wherein the method comprises incubating the culture medium for an incubation period after placing the microwells over the array of magnet pairs.

11. (canceled)

12. The method of claim 1, wherein the paramagnetic culture medium is prepared using a paramagnetic salt solution.

13. The method of claim 1, wherein the microwells have synthetic and/or natural extracellular matrix (ECM) coated well surfaces.

14. (canceled)

15. The method of claim 1, wherein the cells are selected from at least one of adherent cell lines, non-adherent cell lines, primary cells, and stem cells.

16. The method of claim 1, wherein the cells comprise different cell types and/or different cell lines and are adapted to be co-cultured.

17. The method of claim 1, wherein the method comprises forming the cellular aggregate with more than one layer of cells using similar or different cell types.

18. The method of claim 17, wherein the method comprises forming layers of the cellular aggregate on top of one another so that the layers of the cellular aggregate are adjacent to one another or separated by a natural ECM or a synthetic ECM.

19. The method of claim 1, wherein the magnets comprise manufacturing tolerances of about ±0.1 mm for creating cellular aggregates with uniform cell-free areas having increased reproducibility to facilitate high throughput assays.

20. The method of claim 1, wherein the method comprises generating cellular aggregates for use in cell migration assays, 3D cell invasion assays, wound healing assays, study cell interactions, drug discovery and drug screening, and/or disease models, where the disease models include asthma models and/or Inflammatory Bowel Disease models.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. A kit comprising a paramagnetic agent, and an array of magnet pairs for applying an external magnetic field gradient to at least one receptacle, wherein the magnet pairs comprise an outer magnet and an inner magnet that is disposed within the outer magnet with a spaced region there between.

37. (canceled)

38. The kit of claim 36, wherein the kit further comprises spacers that are shaped for placement within the magnet pairs.

39. The kit of claim 36, wherein the kit further comprises a cell culture plate, a cuvette or a micro titer plate that provides the at least one receptacle.

40. An apparatus for generating cell-free regions in a cellular aggregate using label-free magnetic manipulation, wherein the apparatus comprises:
a plurality of microwells that are seeded with cells in a paramagnetic culture medium to create a magnetic susceptibility difference, the cells being diamagnetic; and
an array of magnet pairs where each magnet pair creates a magnetic field gradient, wherein the plurality of microwells with the cells and paramagnetic culture medium are disposed over the array of magnet pairs, and
wherein the magnetic susceptibility difference and the magnetic field gradient drive the cells towards a spaced region between magnets in the magnet pair having a lowest magnetic field strength to create the cell aggregate with a cell-free area where the cell aggregate surrounds the cell free-area.

41. (canceled)
